(19) United States
(12) Patent Application Publication
(10) Pub. No.: US 2024/0309307 A1
(43) Pub. Date: Sep. 19, 2024

(54) Super Nozzle Injection Fermentor

(71) Applicant: Unibio Tech Science A/S, Odense SØ (DK)

(72) Inventor: Ebbe Busch LARSEN, Odense SØ (DK)

(73) Assignee: Unibio Tech Science A/S, Odense SØ (DK)

(21) Appl. No.: 18/272,643

(22) PCT Filed: Feb. 3, 2022
(86) PCT No.: PCT/DK2022/050017

§ 371 Date: Jul. 17, 2023

(30) Foreign Application Priority Data
Feb. 4, 2021 (DK) PA202170054

(51) Int. Cl. C12M 1/00

(52) U.S. Cl. CPC C12M 29/06; C12M 29/18; C12M 29/20

(57) ABSTRACT

A fermentor system for cultivation of microorganisms dependent on gas supply for growth includes an inlet pipe feeding growth medium and gas connected with one or more adjustable inlet pump members allowing a given velocity of growth medium and gas through the inlet pipe; a fermentor tank having a cylindrical volume, the fermentor tank including liquid media with a microorganism to be cultivated and a plurality of upper feeding nozzles that receive and distribute growth medium and gas from the inlet pipe into the fermentor tank; an inner cylindrical outlet pipe, extending from the bottom to the top of the fermentor tank, and being encircled by the plurality of upper feeding nozzles at the top of the fermentor tank; and a transport pipe transporting liquid media from the outlet pipe to a dewatering separator, thereby obtaining concentrated living microorganism material for further processing and liquid separated from concentrated living microorganism material.

### FIELD OF THE INVENTION

The present invention relates to a system and a method for fermentation of microorganisms that are dependent on gas supply for growth. In particular, the invention is useful for fermentation of bacteria, such as methylotrophic or methanotrophic bacteria.

### BACKGROUND OF THE INVENTION

Traditionally, fermentor systems have been applied for cultivation of microorganisms in aqueous solutions containing a variety of growth media, including carbon sources and nitrogen sources. Numerous fermentor systems require presence of a gas in the fermentation media, such as oxygen.

Oxygen may be applied by pumping compressed atmospheric air into the fermentation media. Pure gaseous oxygen or oxygen enriched air may also be used as an oxygen source. In other systems, other gases may be applied in combination with oxygen, such as methane. Typically, methane is used when methylotrophic or methanotrophic bacteria are to be fermented. Waste products are formed during fermentation, such as carbon dioxide.

Generally, problems may occur in liquid fermentation systems where microorganisms are dependent on gas supply for growth since the microorganisms usually cannot use the gases directly. Accordingly, in order to provide an effective system, the gases need to be dissolved in the fermentation liquid. Particularly, there is a need for appropriate dissolution of gases in these systems in order to comply with the demands of the microorganisms, particularly if the population or concentration of microorganisms is large, or if the fermentation temperature is high.

In commercially available production units, there is a need for fermentation systems that may accommodate a large turnover of microorganisms and thereby the provision of highly effective and reliable systems for cultivation of microorganisms that are dependent on gas supply for growth. The transfer rate of substances from the gas phase into the liquid phase can be improved if very small bubbles are generated, or if a higher pressure is used in the fermentor.

Conventional systems with stirring blades have been used in the past to serve an appropriate mixing of gases with fermentation liquid. Typically, these systems are made by adding gases at the bottom of a tank under pressure. This compression of gases requires significant amounts of energy.

Other fermentor types have also been designed with the intention of reducing energy consumption for mixing but still ensuring sufficient mass transfer of gases to the liquid phase. These fermentors are often called air lift fermenters, jet loop fermenters loop fermenters, or U-loop fermentors.

Although, U-shape fermentors have been shown to provide for a long contact time between the gas and liquid phases, there is a need in the art for systems that are more effective in accommodating gas to liquid transition, and dispersion of gas in the fermentation liquid. The transfer of gases between the gas phase and the liquid phase in such fermenters is still too poor for producing inexpensive products in a fermentation process.

Likewise, residence time and concentration of gases in the liquid is very important. For instance, a higher pressure in the fermenter may reduce the release of gases, such as carbon dioxide, to the gas phase in the fermentor, resulting in a higher concentration of gases, such as carbon dioxide, in the fermentation broth. A high concentration of carbon dioxide in the fermentation liquid may cause a reduced productivity of the cells in the fermentor and thereby a reduced overall productivity in the fermentation process.

Accordingly, an appropriate flow through the fermentation system is of importance in addition to various other parameters, such as optimum gas to liquid transition. Therefore, there is still a need in the art for further improvement of the overall productivity of fermentors and fermentation processes and especially further improvement of the utilization of the substrate gases added to such fermentations.

### SUMMARY OF THE INVENTION

Accordingly, there is provided a fermentor system for cultivation of microorganisms that are dependent on gas supply for growth, the system comprising:
an inlet pipe for feeding of growth medium and gas, the inlet pipe having a diameter (D₁) and being connected with one or more adjustable inlet pump members allowing a given velocity (V₁) of growth medium and gas through the inlet pipe;
a fermentor tank having a substantially cylindrical volume and a diameter (D₂), the fermentor tank including liquid media with a microorganism to be cultivated and a plurality of upper feeding nozzles that receive and distribute growth medium and gas from the inlet pipe into the fermentor tank as a function of V₁ and thereby allowing a given velocity (V₂) of liquid media from top to bottom of the fermentor tank;
an inner cylindrical outlet pipe having a diameter (D₃), extending from the bottom to the top of the fermentor tank, and being encircled by the plurality of upper feeding nozzles at the top of the fermentor tank, allowing a given velocity (V₃) of liquid media from bottom to top through the inner cylindrical outlet pipe that is higher than V₂ driven by the flow of liquid media through the fermentor system; and
a transport pipe having a diameter (D₄) less than D₃ and a velocity (V₄) higher than V₃ for transporting liquid media from the outlet pipe to a dewatering separator, and thereby obtaining concentrated living microorganism material for further processing and liquid separated from the concentrated living microorganism material.

Additionally, there is provided a method of cultivating microorganisms that are dependent on gas supply for growth in a fermentor system, the method comprising the steps of:
feeding of growth medium and gas through an inlet pipe, the inlet pipe having a diameter (D₁) and being connected with one or more adjustable inlet pump members allowing a given velocity (V₁) of growth medium and gas through the inlet pipe;
cultivating a microorganism in a fermentor tank having a substantially cylindrical volume and a diameter (D₂), the fermentor tank including liquid media with a microorganism to be cultivated and a plurality of upper feeding nozzles that receive and distribute growth medium and gas from the inlet pipe into the fermentor tank as a function of V₁ and thereby allowing a given velocity (V₂) of liquid media from top to bottom of the fermentor tank;
allowing the liquid media to enter an inner cylindrical outlet pipe having a diameter (D₃), extending from the bottom to the top of the fermentor tank, and being encircled by the plurality of upper feeding nozzles at the top of the fermentor tank, allowing a given velocity (V₃) of liquid media from bottom to top through the inner cylindrical outlet pipe that is higher than V₂ driven by the flow of liquid media through the fermentor system; and
conveying the liquid media through a transport pipe having a diameter (D₄) less than D₃ and a velocity (V₄) higher than V₃ from the outlet pipe to a dewatering separator, and thereby obtaining concentrated living microorganism material for further processing and liquid separated from the concentrated living microorganism material.

### BRIEF DESCRIPTION OF DRAWINGS

The invention will be understood in greater detail with reference to the following figures that serve to illustrate certain particular embodiments of the invention by way of example:

FIG. 1 illustrates embodiments of the invention, including an inlet pipe having a diameter (D₁), a fermentation tank having a diameter (D₂), a plurality of nozzles located in the top of the tank, here illustrated as a circular wreath-shaped member with numerous tubes extending from an inner ring-formed portion in the end of the inlet pipe, an outlet pipe having a diameter (D₃), with an opening in the bottom of the tank allowing liquid to exit the tank centrally in the tank, and a transport pipe having a diameter (D₄) for transporting the liquid to a separator for further processing. A pump is illustrated providing the system with gas and growth medium.

FIG. 2 illustrates embodiments of the invention, where concentrated living microorganism material are conveyed for further processing to a feed mixing station, and further to a storage tank for enrichment of feed.

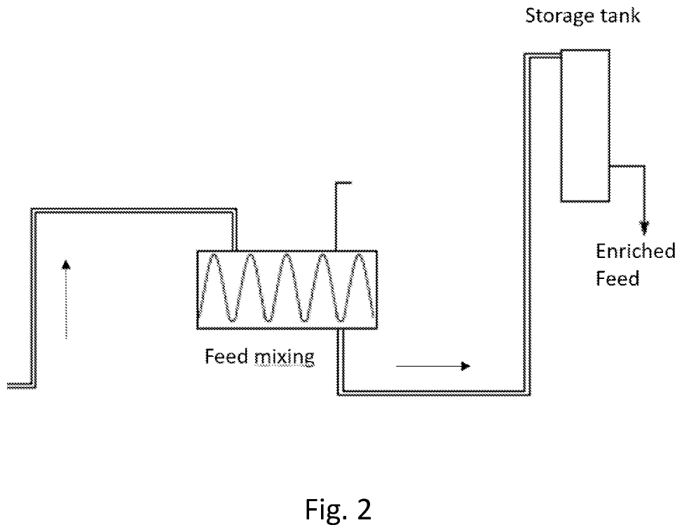

### DETAILED DESCRIPTION

The fermentor system and method of the present invention are associated with several advantages that serve to improve the fermentor systems of the prior art.

Generally, the transfer of gases between the gas phase and the liquid phase in the system and method of the invention is improved compared to conventional fermentor systems and may result in production of less expensive products in the fermentation process. At the same time, an appropriate flow through the fermentation system may provide an appropriate retention time through the system and indirectly also improved gas to liquid transition, resulting in improvement of the overall productivity and especially further utilization of the substrate gases added to such fermentations.

The dimensions of the fermentor system of the present invention may serve an appropriate flow through the system and optimum conditions for growth of microorganisms, such as bacteria. Dependent on the microorganism to be fermented, an optimum supply of growth medium and gas into the fermentation liquid may be complemented with an optimum flow through the system in order to get rid of waste products from the fermentation process that may compromise growth. In that way, an optimum steady state equilibrium may be obtained for optimum production of microorganisms where interference from waste products are limited and at the same time growth medium and gas supply are added in a concentration or quantity that results in the best conditions for growth of the microorganisms.

For instance, the diameter (D₂) of the fermentation tank may be dimensioned to provide a certain velocity (V₂) of fermentation liquid in a downward direction as a function of the supply of growth medium and gas in order to establish the best conditions for growth of the microorganisms. At the same time, the production of waste products that may impact or suppress growth may be accommodated for by the velocity (V₂).

Likewise, the diameter (D₃) of the outlet pipe may be dimensioned to provide a certain velocity (V₃) of fermentation liquid out of the system in an upward direction in order to establish the best conditions for growth of the microorganisms. Since the diameter (D₃) of the outlet pipe is smaller than the diameter (D₂) of the fermentation tank, the velocity (V₃) of fermentation liquid out of the system is higher. This is used to provide an optimum flow for the operation of the fermentation system and method according to the invention. Since fermentation still occurs in the outlet pipe, the diameter (D₃) of the outlet pipe may optimize the overall fermentation production in the fermentation system and method.

Turning to the transport pipe having a diameter (D₄), the diameter is dimensioned to provide a certain velocity (V₄) of fermentation liquid that is even higher than (V₃), i.e., having a diameter that is usually considerably smaller than the diameter (D₃). Hence, the fermentation liquid may be readily conveyed to one or more separators that separate liquid from microbial material.

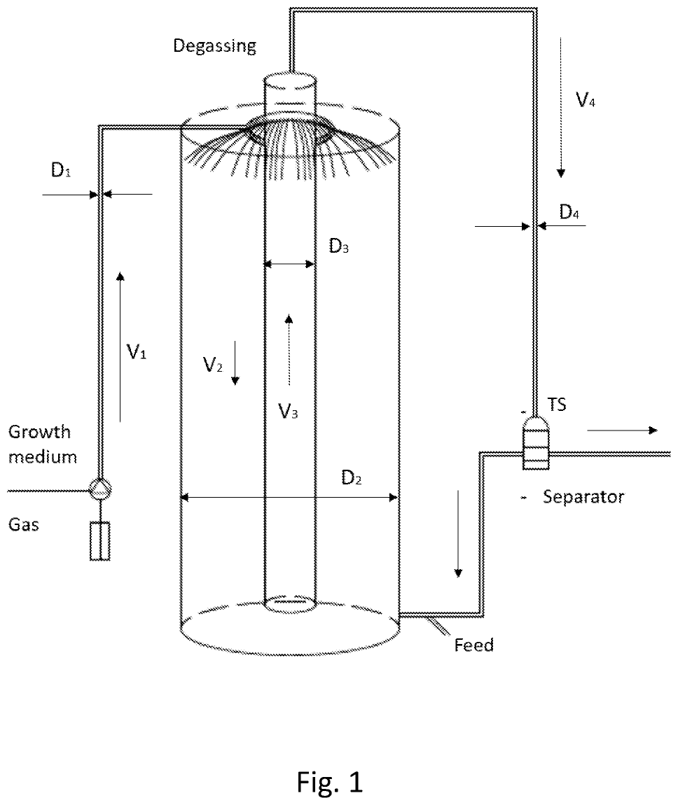

In the present context, the fermentation system is applied in an upraised position as illustrated in FIG. 1. The flow downward in the tank and supply of growth medium and gas in top of the tank together with the upward movement of fermentation liquid out of the system inside the tank may serve various advantages according to the invention. Optimum production and growth are seen as some of the advantages. At the same time, it is important to establish a system that is as simple as possible and at the same time provides the highest production capacity.

Typically, the fermentation system is operated by one or more pump members in connection with the inlet pipe. The one or more pump members may drive the system and the flow through the fermentation tank as well as the outlet pipe and can do so with use of mixing blades in the fermentation tank or outlet pipe. However, further pump member may be present in the system in order to drive the flow of liquid.

Importantly, according to the invention there is provided a plurality of upper feeding nozzles that receive and distribute growth medium and gas from the inlet pipe into the fermentor tank as a function of V₁ and thereby allowing a given velocity (V₂) of liquid media from top to bottom of the fermentor tank. One of the advantages of the plurality of nozzles is that the gas to liquid transition may be optimized for superior growth of the microorganisms, such as for methylotrophic and/or methanotrophic bacteria.

By providing an appropriate pressure by the one or more pump member connected to the inlet pipes, and thereby a certain velocity V₁ in the inlet pipe, small bubbles may be generated in the fermentation liquid of the fermentor tank. Optimization of the presence of bubbles may be adjusted by the pressure applied to the nozzles, and thereby appropriate dissolution of gases in the liquid medium. This embodiment is particularly beneficial according to the invention as the gas to liquid transition is traditionally associated with a limiting factor for growth of microorganisms, such as methylotrophic and/or methanotrophic bacteria.

According to the invention, the system comprises an inlet pipe for feeding of growth medium and gas, the inlet pipe having a diameter (D₁) and being connected with one or more adjustable inlet pump members allowing a given velocity (V₁) of growth medium and gas through the inlet pipe. In the present context, the intended meaning by “growth medium” is a source of nutrients, minerals, phosphate source, carbon source, nitrogen source, etc., as appropriate and known by a person skilled in the art, and dependent on the microorganism to be fermented.

If for instance a methanotrophic bacteria is to be fermented that may metabolize carbon in methane, an organic carbon source is not required in some embodiments of the invention. On the other hand, gases alone usually cannot always provide the necessary building blocks of microorganisms, and therefore “growth medium” is commonly applied. In case of methanotrophic bacteria that may metabolize methane and certain other gases, the “growth medium” would be adjusted accordingly with nutrients and/or minerals.

In some embodiments of the invention, the nitrate concentration of the fermentation liquid during fermentation is in the range of 0-0.035 g/l; e.g. in the range of 0.001-0.033 g/l; such as in the range of 0.002-0.03 g/l; e.g. in the range of 0.003-0.025 g/l; such as in the range of 0.004-0.02 g/l; e.g. in the range of 0.005-0.015 g/l; such as in the range of 0.007-0.01 g/l.

In some embodiments of the present invention, the content of undissolved oxygen in the fermentation reactor or the content of gaseous oxygen present in an exhaust gas may be at most 10% (vol/vol), such as at most 8% (vol/vol), e.g., at most 6% (vol/vol), such as at most 4% (vol/vol), e.g., at most 2% (vol/vol), such as at most 1% (vol/vol), e.g., at most 0.5% (vol/vol), such as 0% (vol/vol).

In some embodiments of the invention, the ratio between C1-C5 carbon source, e.g. methane, and oxygen in the undissolved gas, and/or in the exhaust gas may be at least 5:1 (vol C1-C5 carbon source/vol oxygen), such as at least 6:1 (vol C1-C5 carbon source/vol oxygen), e.g. at least 7:1 (vol C1-C5 carbon source/vol oxygen), such as at least 8:1 (vol C1-C5 carbon source/vol oxygen), e.g. at least 9:1 (vol C1-C5 carbon source/vol oxygen), such as at least 10:1 (vol C1-C5 carbon source/vol oxygen), e.g. at least 15:1 (vol C1-C5 carbon source/vol oxygen), such as at least 20:1 (vol C1-C5 carbon source/vol oxygen), e.g. at least 25:1 (vol C1-C5 carbon source/vol oxygen), such as at least 30:1 (vol C1-C5 carbon source/vol oxygen), e.g. at least 35:1 (vol C1-C5 carbon source/vol oxygen).

During fermentation it may be important to differentiate between dissolved gasses and undissolved gasses, since only the dissolved gases are consumable to the microorganisms. Undissolved gasses, like undissolved C1-C5 carbon source, like methane, and/or undissolved oxygen, which are to be separated in the exhaust gas and may be wasted. In the present context, the term “dissolved” relates to gas, which is absorbed by the fermentation medium, in the present context, gas which has been absorbed by the fermentation medium and become available for consumption by the microorganisms to be cultivated. In contrast to the dissolved gas, there are the undissolved gas. In the present context, the term “undissolved” relates to gas which has not been absorbed by the fermentation medium and will not be available to be consumed by the microorganisms to be cultivated.

With respect to the gases, suitable gases may include oxygen and/or methane, and in other embodiments compressed air or ambient air having a content various gases may be suitable. Most preferred is methane when methylotrophic and/or methanotrophic bacteria are to be fermented. Equally preferred is oxygen. Other gases such a ammonia may also be fermented according to the invention, and may partly constitute the nitrogen source according to the invention.

The pressure applied by the one or more pump members according to the invention in the inlet pump and correspondently to the plurality of nozzles, may be adjusted dependent on the microorganism to be fermented. Here, the dimension of the inlet pipe is important as output of the plurality of nozzles is a function of the velocity (V₁) through the inlet pipe. For instance, if a high pressure is present in the inlet pipe, the nozzles may produce bubbles in the fermentation media and may serve to dissolve more gas in the fermentation media.

In the present context, “one or more pump member” or similar wording is intended to mean that more than one pump may be applied according to the invention. The one or more pump members may be adjusted to provide a suitable pressure and velocity through the inlet pipe. However, also the nozzles size opening may be adjusted to deliver a desirable amount and concentration of gases to the fermentation medium. Additionally, the number of nozzles may be changed according to requirements for a specific production of a specific microorganism.

Generally, the fermentation system is suitable for production of methanotrophic bacteria but may also be suitable for production of other microorganisms. For instance, production of recombinant bacteria for medical purposes may be conducted in the system. In these cases, proteins, peptides, mRNA or DNA may be duplicated and later isolated for further use. In this case, microorganisms may be used as vehicles for multiplication of these subjects. Also, production of yeast may be possible with a suitable set-up.

The fermentor tank according to the invention has a substantially cylindrical volume and a diameter (D₂), the fermentor tank including liquid media with a microorganism to be cultivated and a plurality of upper feeding nozzles that receive and distribute growth medium and gas from the inlet pipe into the fermentor tank as a function of V₁ and thereby allowing a given velocity (V₂) of liquid media from top to bottom of the fermentor tank. In the present context “liquid media” is intended to mean fermentation broth or similar, i.e., media containing a microorganism to be cultivated and the necessary nutrients, minerals, etc. necessary for the microorganism to grow, supplied through the inlet pipe. Liquid, such as water, may be added to keep a steady liquid volume. “Liquid media” is also called fermentation liquids, fermentation broths, or simply broths, containing a variety of substrates, such as carbon sources as well as nitrogen sources, phosphates, sulphates, as well as a wide variety of other components depending on the microorganism used and the products to be made. In many cases, the generic name fermentation is used for such processes, which may be carried out in the presence or the absence of oxygen or air.

The tank is substantially cylindrical, which means that the flow through the system is given by diameters of the pipes, tubes, etc. However, the top of the tank may have a concave or convex shape, whatever is appropriate for a specific production. Also, the bottom of the tank my be concave in order to avoid sedimentation of microorganisms around the circular edges. The cylindrical form may give an advantage to control the flow in the system and the velocities involved through the pipes and cylinders. The concentration and amount of microorganisms may preferably be in steady state and in equilibrium in the system.

In the present context, a “fermentor tank” or similar wording is intended to mean a vessel suitable for conducting fermentation or for employing biocatalysts. A fermentation process is defined as the growth or maintenance of living biocatalysts under aerobic, anaerobic, or partially aerobic conditions such that a desired product is produced, whether that product is the cells themselves or substances produced by the cells or converted by the cells. Living biocatalysts encompass microbial cells, animal cells, insect cells, plant cells, viruses, phage, prions, amoebae, algae, fungi, bacterial, prokaryotic, or eukaryotic cells. Non-living biocatalysts are dead cells or extracts from living or dead cells, e.g., enzymes.

By adding a constant supply of growth medium and gases, allowing waste gases to escape from the system and harvesting organic material from the system, a steady state may be achieved. By configuring the system in accordance with the invention, a higher concentration of microorganisms may be established compared to conventional systems, such as U-shaped fermentors. Also, a much higher production of organisms may be produced in a comparable lower volume of liquid in view of conventional methods. In turn, this results in a more efficient system and lower costs associated with the production of microorganisms.

Importantly, the plurality of nozzles used in the system accommodated a higher level of gas to liquid transition compared to conventional systems. The number of nozzles may be adjusted to the specific microorganism to be produced, and may be 10, 20, 50, or even higher. The number of nozzles may to a high degree reflect the effectivity in dissolving gases in the liquid medium. However, also the pressure subjected to the nozzles may impact the effectivity in dissolving gases in the liquid medium. Preferably, the one or more pump members are used to regulate the generation of bubbles in the liquid medium. But also, the number and size of nozzle openings may be used to further adjust the system.

During initialization of the system, a strain of microorganism (e.g., inoculum) is supplied in the liquid medium. Preferably, this strain is a bacteria strain of a specific origin. During operation, this strain grows exponentially and becomes dominant in the system, and usually outcompeting other microorganisms until a steady state is reached and equilibrium is obtained. Typically, initialization is made by only one strain of a certain origin, and other strains are strictly avoided. In some cases, other strains may not be avoided, and in that case certain means may be applied to limit growth of these other strains. In some other cases, the target strain to be cultivated outcompetes these other strains. Usually, it is the aim that only one strain of microorganism is cultivated at a time. However, in some embodiments “a microorganism” may refer to “one or more microorganisms”.

The term “inoculation” or similar wording refers to the placement of microorganisms (e.g., methane producing bacteria) that will grow when implanted in a culture medium, such as a fermentation tank comprising media to be fermented. “Inoculum” refers to the material used in an inoculation, for example a composition comprising microorganisms, which is employed to prime a process of interest. For example, an inoculum where the bacteria is essentially methane producing bacteria may be used to direct a methanotrophic formation process in a culture medium in a fermentation tank comprising said media (e.g., a feed product).

Thus, “to inoculate” refers to the transfer of the inoculum to the media to be processed, for example, the transfer of the inoculums to a proteinaceous feed material to be fermented. The primary inoculum refers to the generation of the initial inoculum in a series of repeated similar of essentially identical inoculation process, for example one or more repetitions of a fermentation process. An aliquot of the product of the formation process may be used to inoculate a new process of fermentation. Thus, the inoculation may be a fermented feed product, which comprises viable methane producing bacteria in sufficient amount to prime a methanotrophic fermentation process of another proteinaceous feed material to be fermented. The inoculum may be a in a liquid form, dry form, or essentially dry form. The moisture % of the inoculum may be adjusted in order to optimize the fermentation process. Thus, the inoculum used in the processes of the present invention may be a fermented feed product.

In one or more embodiments, the inoculum is provided as essentially pure viable bacteria (such as bacteria in freeze dried form) or bacteria suspended in a suitable media prior to the application (such as a water, buffer, or a growth media).

The fermentation process can be controlled by varying e.g., temperature and time to optimize the fermentation reaction. Thus, in some embodiments of the invention, the temperature is within the range of 15-45° C., such as 15-40° C., such as 25-35° C., such as 30-40° C., such as 15-20° C., or such as 40-45° C.

In some embodiments of the invention, methanotrophic bacteria may be added as an inoculum comprising essentially methanotrophic bacteria and/or an isolated methanotrophic bacteria or spore. Accordingly, in one embodiment of the invention, the proportion of said added inoculum comprising essentially methanotrophic bacteria in the bacterially enriched animal feed composition is within the range of 0.1-99.9 vol-%, 1-99 vol-%, 5-95 vol-%, 10-90 vol-%, 15-85 vol-%, 20-80 vol-%, 25-75 vol-%, 30-70 vol-%, 35-65 vol-%, 40-60 vol-%, 45-55 vol-%, preferably around 1-5 vol-%, such as 2-4 vol-%. Thus, the inoculum is provided with a concentration of methanotrophic bacteria sufficient to reduce amount of methane emanating from the digestive tract of ruminants (or livestock).

Methylotrophic and/or methanotrophic bacteria are preferred. A person skilled in the art would know which strains may be used. These include bacteria are selected from the group consisting of Methylomonas, Methylobacter, Methylococcus, Methylosinus, and mixtures thereof. Preferably, Methylococcus capsulatus. In a further embodiment of the present invention the one or more aerobic methanotrophic bacteria may comprises a combination of M. capsulatus (preferably NCIMB 11132)) A. acidovorans (preferably NCIMB 13287); B. firmus (preferably NCIMB 13289); and A danicus (preferably NCIMB 13288).

When methylotrophic and/or methanotrophic bacteria are fermented, the following two equations apply:

CH₄+1.22 0₂+0.104NaNOs->0.52 Biomass+0.48C0₂+1.532 H₂0 (1)

CH₄+1.45 0₂+0.104NH₃->0.52 Biomass+0.48C0₂+1.69 H₂0 (2)

Thus, these reactions require both methane and oxygen together with either nitrate or ammonia. A standard stoichiometry, as shown in formulas (1) and (2) above, illustrates why the high concentrations of oxygen relative to the concentration of methane is to be used. From the standard stoichiometric point of view the demand of oxygen is higher than the demand for methane in order to provide the desired biomass product, where methane react with oxygen and a nitrogen source. In one embodiment of the invention, the gas comprises a C1-C5 carbon source, such as methane.

The system and method according to the invention may include one or more sensors, such as gas sensors, to control the level of growth medium and gas supplied in the system. But also to monitor the system and conditions in the fermentation tank, outlet pipe and/or transport pipe.

According to the invention, the system includes an inner cylindrical outlet pipe having a diameter (D₃), extending from the bottom to the top of the fermentor tank, and being encircled by the plurality of upper feeding nozzles at the top of the fermentor tank, allowing a given velocity (V₃) of liquid media from bottom to top through the inner cylindrical outlet pipe that is higher than V₂ driven by the flow of liquid media through the fermentor system.

Additionally, the system according to the invention includes a transport pipe having a diameter (D₄) less than D₃ and a velocity (V₄) higher than V₃ for transporting liquid media from the outlet pipe to a dewatering separator, and thereby obtaining concentrated living microorganism material for further processing and liquid separated from the concentrated living microorganism material.

In the present context, “concentrated living microorganism material” or similar wordings is intended to mean living cells separated from liquid, such as water, for further use. For the avoidance of doubt, this material may comprise dead microorganisms to a certain limited degree, but predominantly living material. In some embodiment of the invention, the content of living cells in the “concentrated living microorganism material” is more than 80% (w/w), such as more than 90% (w/w), such as more than 95% (w/w). The dewatering separation may be a centrifuge, and liquid separated from the concentrated living microorganism material may be a supernatant, i.e., the liquid fractioned in top of a vessel, contrary to precipitate in a suspension. The “concentrated living microorganism material” may also be denoted “precipitate”.

In some embodiments of the invention, the concentrated living microorganism material is further conveyed to one or more feed mixers where it is blended with feed ingredients to constitute about 2-20% (w/w) of microorganism enriched feed, such as 3-15% (w/w), such as 5-10% (w/w). These feed ingredients may be maize or other suitable and cheap feed being part of conventional feed to animals. Hence, feed is added in some embodiment of the invention in an amount of about 80-98% (w/w) of microorganism enriched feed, such as 85-97% (w/w), such as 90-95% (w/w).

In some embodiments of the invention, mixing time with feed is from 10 second to 20 minutes, such as from 20 second to 10 minutes, such as from 30 second to 10 minutes. The feed may be further conveyed to a storage tank where it is kept at a certain temperature, such from −15 to 5 Degrees Celsius in order to avoid damages to the feed and to avoid that the feed does not freeze to ice. This enriched feed may be transported to the animals or used directly.

One of the great benefits of the invention is that living microorganism may be included in feed for animals, such as domestic livestock. This may be helpful in order to supplement nutrients, proteins, and the like in the animal feed, but may also accommodate increased digestion, alleviate certain less preferred conditions in animals or contribute to a lower production of certain gases from animals. For instance, if methylotrophic and/or methanotrophic bacteria is added to the animal fees, less methane production may be achieved, balancing the requirements of emission of greenhouse gasses. Domestic livestock may include cattle, buffalo, sheep, goats, and camels. These animals produce large amounts of methane as part of their normal digestive process. In addition, methane is produced when animals' manure is stored or managed in lagoons or holding tanks.

In some embodiments of the invention, the concentrated living microorganism material is supplied to animals for reducing methane production.

In some embodiments of the invention, the liquid separated from the concentrated living microorganism material is recycled to the fermenter tank, preferably to the bottom of the fermentor tank through a recycling pipe. However, the liquid may also be supplied in top of the tank or other places.

In some embodiments of the invention, the microorganism is a methylotrophic and/or methanotrophic bacteria. In some embodiments of the invention, the microorganism is an aerobic microorganism. In some embodiments of the invention, the aerobic microorganism is an aerobic methanotrophic microorganism and/or one or more aerobic methylotrophic microorganism. In some embodiments of the invention, the one or more aerobic methanotrophic microorganism or one or more aerobic methylotrophic microorganism is one or more aerobic methanotrophic bacteria and/or one or more aerobic methylotrophic bacteria. In some embodiments of the invention, the one or more aerobic methanotrophic bacteria is selected from a Methylococcus.

In some embodiments of the invention, the gas in the inlet pipe comprises oxygen and/or methane for cultivation of the microorganism that are dependent on gas supply for growth.

In some embodiments of the invention, the plurality of upper feeding nozzles each comprises a tube of a certain length extending down into the liquid media of the fermentor tank, and wherein pressure subjected to the nozzles accommodates numerous bobbles in the liquid media.

In some embodiments of the invention, the plurality of upper feeding nozzles are adjusted to provide an optimum gas to liquid transition in the fermentor tank depending on the microorganism being fermented.

In some embodiments of the invention, the diameters D₁, D₂, D₃ and D₄ are dimensioned to provide an optimum fermentor system depending on the microorganism being fermented. In some embodiments of the invention, the velocities V₁, V₂, V₃ and V₄ are adjusted to provide an optimum fermentor system depending on the microorganism being fermented.

In some embodiments of the invention, the system further comprising a degassing valve positioned at the end of the inner cylindrical outlet pipe on top of the fermentor tank allowing gasses to escape from the liquid media.

In some embodiments of the invention, the bottom of the fermentor tank is concave in order to minimize sedimentation around the outer areas of the circular bottom.

In some embodiments of the invention, the dewatering separator is operable to provide a liquid content of 3-50% (w/w), such as 5-40% (w/w), such as 8-35% (w/w) of the concentrated living microorganism material.

In some embodiments of the invention, the fermentor system comprises means for further processing the concentrated living microorganism material into amino acids, peptides and/or proteins for enrichment of animal feed. In some cases, living organisms is not the direct target and the enriched feed may be further treated to extract amino acids, peptides and/or proteins for enrichment of animal feed. This may also occur in a parallel track with the track to obtain living microorganisms to the animal feed. An amino acid profile may be obtained that satisfies the needs for animals, where essential amino acids are present in abundance. The enriched feed may also be used for fish food.

One of the advantages of the invention is to obtain a high production output that may be used to provide supplement amino acids, peptides and/or proteins for enrichment of animal feed. Another advantages of the invention is to obtain a high production output that may be used to provide living microorganisms to animal feed. Yet another advantage is to obtain both a high production output that may be used to provide supplement amino acids, peptides and/or proteins for enrichment of animal feed and to obtain a high production output that may be used to provide living microorganisms to animal feed.

In some embodiments of the invention, the microorganism is a recombinant microorganism and the fermentor system is applied to cultivate microorganisms for medical purposes. In some embodiments, the one or more microorganism does not include a recombinant microorganism. In the context of the present invention the term “recombinant microorganism” relates to a genetically modified organism (GMO) whose genetic material has been altered using plasmids, deletion of existing genes; or other genetic engineering techniques. The recombinant microorganism may be considered in contrast to genetic alterations that occur naturally in the microorganism, e.g., by mating and/or natural mutation.

In another aspect of the invention, there is provided a method of cultivating microorganisms that are dependent on gas supply for growth in a fermentor system, the method comprising the steps of:
feeding of growth medium and gas through an inlet pipe, the inlet pipe having a diameter (D₁) and being connected with one or more adjustable inlet pump members allowing a given velocity (V₁) of growth medium and gas through the inlet pipe;
cultivating a microorganism in a fermentor tank having a substantially cylindrical volume and a diameter (D₂), the fermentor tank including liquid media with a microorganism to be cultivated and a plurality of upper feeding nozzles that receive and distribute growth medium and gas from the inlet pipe into the fermentor tank as a function of V₁ and thereby allowing a given velocity (V₂) of liquid media from top to bottom of the fermentor tank;
allowing the liquid media to enter an inner cylindrical outlet pipe having a diameter (D₃), extending from the bottom to the top of the fermentor tank, and being encircled by the plurality of upper feeding nozzles at the top of the fermentor tank, allowing a given velocity (V₃) of liquid media from bottom to top through the inner cylindrical outlet pipe that is higher than V₂ driven by the flow of liquid media through the fermentor system; and
conveying the liquid media through a transport pipe having a diameter (D₄) less than D₃ and a velocity (V₄) higher than V₃ from the outlet pipe to a dewatering separator, and thereby obtaining concentrated living microorganism material for further processing and liquid separated from the concentrated living microorganism material.

This method may be applied with the same means and embodiments as outlined for the system according to the invention.

1. A fermentor system for cultivation of microorganisms that are dependent on gas supply for growth, the system comprising:
an inlet pipe for feeding of growth medium and gas, the inlet pipe having a diameter (D₁) and being connected with one or more adjustable inlet pump members allowing a given velocity (V₁) of the growth medium and the gas through the inlet pipe;
a fermentor tank having a substantially cylindrical volume and a diameter (D₂), the fermentor tank including liquid media with a microorganism to be cultivated and a plurality of upper feeding nozzles that receive and distribute the growth medium and the gas from the inlet pipe into the fermentor tank as a function of V₁ and thereby allowing a given velocity (V₂) of liquid media from a top to a bottom of the fermentor tank;
an inner cylindrical outlet pipe having a diameter (D₃), extending from the bottom to the top of the fermentor tank, and being encircled by the plurality of upper feeding nozzles at the top of the fermentor tank, allowing a given velocity (V₃) of liquid media from the bottom to the top through the inner cylindrical outlet pipe that is higher than V₂ driven by the flow of liquid media through the fermentor system; and
a transport pipe having a diameter (D₄) less than D₃ and a velocity (V₄) higher than V₃ for transporting liquid media from the outlet pipe to a dewatering separator, and thereby obtaining concentrated living microorganism material for further processing and liquid separated from the concentrated living microorganism material.

2. The fermentor system according to claim 1, wherein the concentrated living microorganism material is further conveyed to one or more feed mixers where concentrated living microorganism material is blended with feed ingredients to constitute about 2-20% (w/w) of microorganism enriched feed.

3. The fermentor system according to claim 1, wherein the concentrated living microorganism material is supplied to animals for reducing methane production.

4. The fermentor system according to claim 1, wherein the liquid separated from the concentrated living microorganism material is recycled to the fermenter tank.

5. The fermentor system according to claim 1, wherein the microorganism is a methylotrophic and/or methanotrophic bacteria.

6. The fermentor system according to claim 1, wherein the gas in the inlet pipe comprises oxygen and/or methane for cultivation of the microorganism that are dependent on gas supply for growth.

7. The fermentor system according to claim 1, wherein the plurality of upper feeding nozzles each comprises a tube of a certain length extending down into the liquid media of the fermentor tank, and wherein pressure subjected to the nozzles accommodates numerous bubbles in the liquid media.

8. The fermentor system according to claim 1, wherein the plurality of upper feeding nozzles are adjusted to provide an optimum gas to liquid transition in the fermentor tank depending on the microorganism being fermented.

9. The fermentor system according to claim 1, wherein the diameters D₁, D₂, D₃ and D₄ are dimensioned to provide an optimum fermentor system depending on the microorganism being fermented.

10. The fermentor system according to claim 1, further comprising a degassing valve positioned at an end of the inner cylindrical outlet pipe on top of the fermentor tank allowing gasses to escape from the liquid media.

11. The fermentor system according to claim 1, wherein the bottom of the fermentor tank is concave in order to minimize sedimentation around the outer areas of the bottom.

12. The fermentor system according to claim 1, wherein the dewatering separator is operable to provide a liquid content of 3-50% (w/w) of the concentrated living microorganism material.

13. The fermentor system according to claim 1, wherein the fermentor system comprises means for further processing the concentrated living microorganism material into amino acids, peptides and/or proteins for enrichment of animal feed.

14. The fermentor system according to claim 1, wherein the microorganism is a recombinant microorganism and the fermentor system is applied to cultivate microorganisms for medical purposes.

15. A method of cultivating microorganisms that are dependent on gas supply for growth in a fermentor system, the method comprising the steps of:
feeding of growth medium and gas through an inlet pipe, the inlet pipe having a diameter (D₁) and being connected with one or more adjustable inlet pump members allowing a given velocity (V₁) of the growth medium and the gas through the inlet pipe;
cultivating a microorganism in a fermentor tank having a substantially cylindrical volume and a diameter (D₂), the fermentor tank including liquid media with a microorganism to be cultivated and a plurality of upper feeding nozzles that receive and distribute the growth medium and the gas from the inlet pipe into the fermentor tank as a function of V₁ and thereby allowing a given velocity (V₂) of liquid media from a top to a bottom of the fermentor tank;
allowing the liquid media to enter an inner cylindrical outlet pipe having a diameter (D₃), extending from the bottom to the top of the fermentor tank, and being encircled by the plurality of upper feeding nozzles at the top of the fermentor tank, allowing a given velocity (V₃) of liquid media from the bottom to the top through the inner cylindrical outlet pipe that is higher than V₂ driven by flow of liquid media through the fermentor system; and
conveying the liquid media through a transport pipe having a diameter (D₄) less than D₃ and a velocity (V₄) higher than V₃ from the outlet pipe to a dewatering separator, and thereby obtaining concentrated living microorganism material for further processing and liquid separated from the concentrated living microorganism material.
